(19) United States
(12) Patent Application Publication
(10) Pub. No.: US 2018/0144091 A1
(43) Pub. Date: May 24, 2018

(54) INFORMATION PROCESSING METHOD, INFORMATION PROCESSING SYSTEM, AND RECORDING MEDIUM

(71) Applicant: FUJITSU LIMITED, Kawasaki-shi (JP)

(72) Inventors: Toshiaki FUNAKUBO, Kawasaki (JP); Tatsuya MORI, Sagamihara (JP); Daisuke UCHIDA, Atsugi (JP); Kazuho MAEDA, Kawasaki (JP); Akihiro INOMATA, Atsugi (JP); Yasuyuki NAKATA, Zama (JP)

(73) Assignee: FUJITSU LIMITED, Kawasaki-shi, Kanagawa (JP)

(21) Appl. No.: 15/817,419

(22) Filed: Nov. 20, 2017

(30) Foreign Application Priority Data
Nov. 22, 2016 (JP) 2016-227122

(51) Int. Cl. G06F 19/12; A61B 5/0205; A61B 5/00; A61B 5/11

(52) U.S. Cl. CPC G06F 19/12; A61B 5/0205; A61B 5/024; A61B 5/11; A61B 5/7278; A61B 5/7242

(57) ABSTRACT

An information processing method executed by a processor included in an information processing apparatus coupled to a sensor, the method includes acquiring information on a living body from the sensor; estimating, by using estimation models, indices other than one unknown index among three indices including an index being related to a feature of a change with time of a biological response signal value derived from a meal after completion of the meal, an index regarding energy consumed by the living body in a time period, and an index regarding a change in an amount of energy stored in the living body in the time period, based on the acquired information; estimating the one unknown index based on a relation satisfied by the three indices and a result of the estimating indices; and generating at least one of model parameters of the estimation models based on the estimated three indices.

### CROSS-REFERENCE TO RELATED APPLICATION

This application is based upon and claims the benefit of priority of the prior Japanese Patent Application No. 2016-227122, filed on Nov. 22, 2016, the entire contents of which are incorporated herein by reference.

### FIELD

The embodiment discussed herein is related to an information processing method, an information processing system, and a recording medium.

### BACKGROUND

Regarding technologies of estimating indices for meal intake, for example, a technology of paying attention to a rise in heart rate during a meal to estimate the amount of the meal has been disclosed (see, for example, Japanese Laid-open Patent Publication No. 10-504739 and the like). Further, a technology of determining calorie intake from body composition and caloric expenditure has been disclosed (see, for example, International Publication Pamphlet No. WO 2013/086363 and the like).

However, with the technologies mentioned above, it is not possible to easily estimate indices regarding energy of a living body. It is desirable that indices regarding energy of a living body may be easily estimated.

### SUMMARY

According to an aspect of the invention, an information processing method executed by a processor included in an information processing apparatus coupled to a sensor, the information processing method includes acquiring information on a living body from the sensor; estimating, by using estimation models, indices other than one unknown index among three indices including an index being related to a feature of a change with time of a biological response signal value derived from a meal after completion of the meal, an index regarding energy consumed by the living body in a predetermined time period, and an index regarding a change in an amount of energy stored in the living body in the predetermined time period, based on information received from the sensor; estimating the one unknown index based on a relation satisfied by the three indices and a result of the estimating indices; and generating at least one of model parameters of the estimation models based on estimated values of the three indices.

The object and advantages of the invention will be realized and attained by means of the elements and combinations particularly pointed out in the claims.

It is to be understood that both the foregoing general description and the following detailed description are exemplary and explanatory and are not restrictive of the invention, as claimed.

### BRIEF DESCRIPTION OF DRAWINGS

FIG. 1 is a diagram schematically illustrating a configuration of an information processing system according to an embodiment;

FIG. 2 is a diagram illustrating a hardware configuration of the information processing apparatus illustrated in FIG. 1;

FIG. 3 is a functional block diagram of the information processing apparatus;

FIG. 4 is a flowchart (1) illustrating a process of the information processing apparatus;

FIG. 5 is a flowchart (2) illustrating the process of the information processing apparatus; and

FIGS. 6A and 6B are diagrams illustrating a second heart-rate peak area.

### DESCRIPTION OF EMBODIMENT

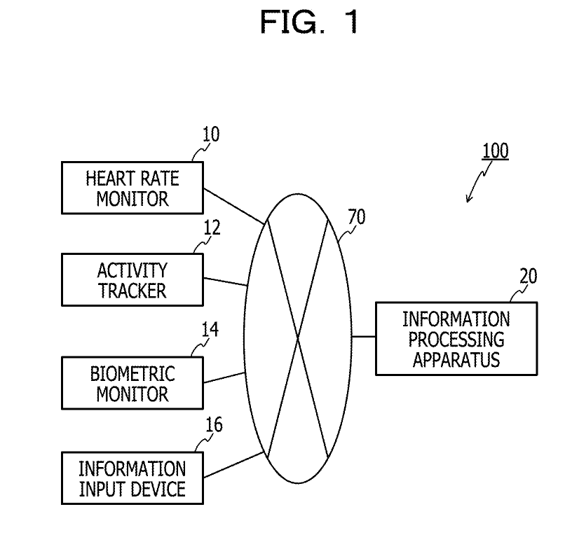

Hereinafter, an embodiment of an information processing apparatus will be described in detail with reference to FIG. 1 to FIGS. 6A and 6B.

FIG. 1 is a diagram schematically illustrating a configuration of an information processing system 100 according to an embodiment. As illustrated in FIG. 1, the information processing system 100 includes a heart rate monitor 10, an activity tracker 12, a biometric monitor 14, an information input device 16, and an information processing apparatus 20. Each device included in the information processing system 100 is coupled to a network 70.

The heart rate monitor 10 is a device for measuring the heart rate (pulse rate) or the like of the user when the device is worn by the user. A measurement result obtained by using the heart rate monitor 10 is transmitted via the network 70 to the information processing apparatus 20.

The activity tracker 12, including an acceleration sensor and the like, is a device for measuring the amount of activity of the user when the device is being held by the user. A measurement result obtained by using the activity tracker 12 is transmitted via the network 70 to the information processing apparatus 20.

The biometric monitor 14 is, for example, a device, including a weight sensor, a bio-impedance sensor, and the like, for measuring the weight and body composition of the user. A measurement result obtained by using the biometric monitor 14 is transmitted via the network 70 to the information processing apparatus 20.

The information input device 16 is, for example, a personal computer (PC) or the like and is a device for the user to enter his or her age, height, initial values of living body quantities, and the like. Information entered to the information input device 16 is transmitted via the network 70 to the information processing apparatus 20. When information such as the age, the height, the initial values of living body quantities, and the like of the user is able to be input from an input unit provided in the information processing apparatus 20, the heart rate monitor 10, the activity tracker 12, or the biometric monitor 14, the information input device 16 may be removed.

The information processing apparatus 20 is, for example, a PC or the like and is a device that estimates indices regarding energy taken in, consumed, or stored by the user based on information acquired from the heart rate monitor 10, the activity tracker 12, the biometric monitor 14, and the information input device 16, and provides an output based on the estimation result. The information processing apparatus 20 may be a smartphone or the like. If any of the heart rate monitor 10, the activity tracker 12, and the biometric monitor 14 directly output energy indices as measured values, estimation processing of the values may be skipped, so that the values are output as is. The information processing apparatus 20 and the heart rate monitor 10 or the like may be coupled to each other without being connected via the network 70.

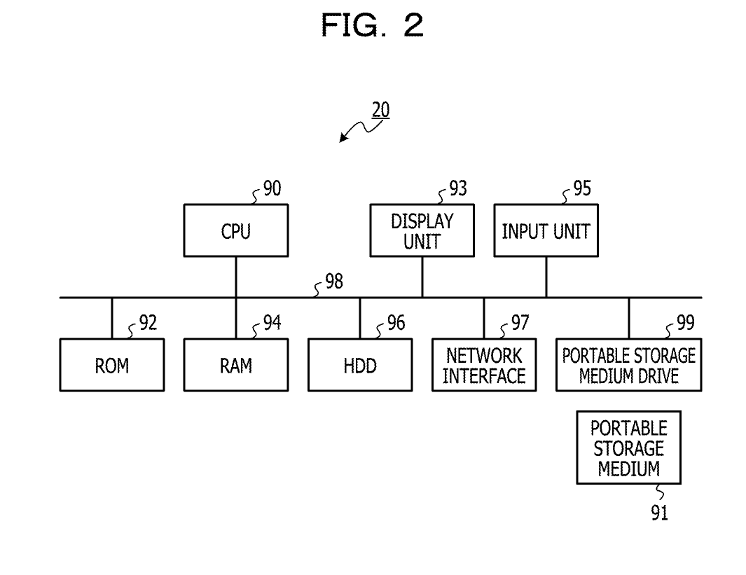

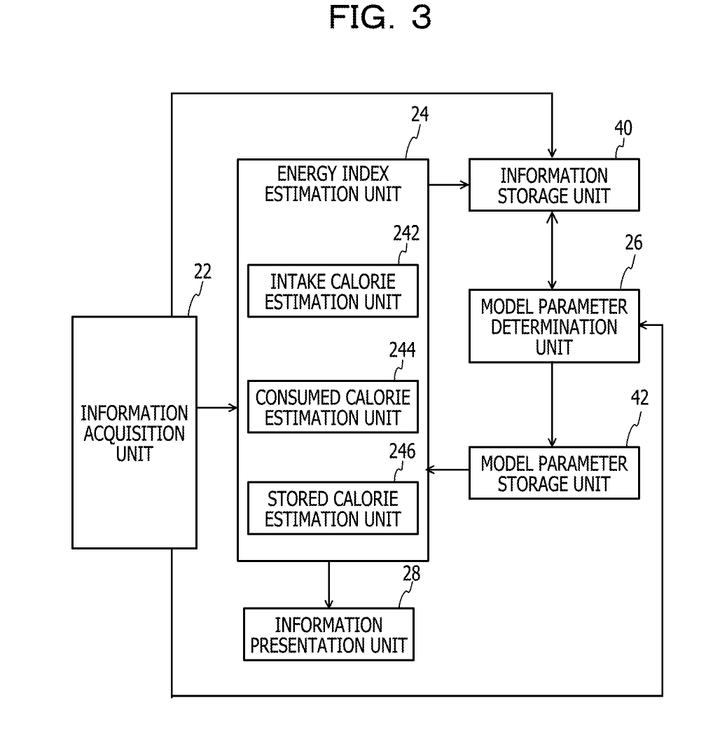

FIG. 2 illustrates a hardware configuration of the information processing apparatus 20. As illustrated in FIG. 2, the information processing apparatus 20 includes a central processing unit (CPU) 90, a read-only memory (ROM) 92, a random access memory (RAM) 94, a storage unit 96, which is here a hard disk drive (HDD), a network interface 97, a portable storage medium drive 99, a display unit 93, an input unit 95, and the like. These component units of the information processing apparatus 20 are coupled to a bus 98. In the information processing apparatus 20, the CPU 90 executes programs (including an information processing program) stored in the ROM 92 or the HDD 96 or programs (including an information processing program) read from a portable storage medium 91 by the portable storage medium drive 99 to achieve the functions of units illustrated in FIG. 3. In FIG. 3, an information storage unit 40 and a model parameter storage unit 42 implemented by the HDD 96 or the like are also illustrated for the sake of illustration.

FIG. 3 illustrates a functional block diagram of the CPU 90 of the information processing apparatus 20. As illustrated in FIG. 3, the CPU 90 functions as an information acquisition unit 22, an energy index estimation unit 24, which is a first estimation unit, a model parameter determination unit 26, which is an generation unit and a second estimation unit, and an information presentation unit 28, which is an output unit, by executing a program.

The information acquisition unit 22 acquires information received from the heart rate monitor 10, the activity tracker 12, the biometric monitor 14, and the information input device 16. Further, the information acquisition unit 22 transmits the information to the energy index estimation unit 24 or the model parameter determination unit 26, and stores the information in the information storage unit 40.

The energy index estimation unit 24 estimates indices regarding energy taken, consumed, and stored by the user. Specifically, the energy index estimation unit 24 includes an intake calorie estimation unit 242, a consumed calorie estimation unit 244, and a stored calorie estimation unit 246 as illustrated in FIG. 3. The intake calorie estimation unit 242 estimates calories taken in a given time period by the user (calories taken in) based on the heart rate of the user acquired from the heart rate monitor 10 in the given time period. The consumed calorie estimation unit 244 estimates calories consumed in a given time period by the user (calories consumed) based on the amount of activity of the user acquired from the activity monitor 12 in the given time period. The stored calorie estimation unit 246 estimates the amount of a change in a given time period in calories stored in the body of the user (a change in the amount of calories stored) based on the weight and the like of the user acquired from the biometric monitor 14 in the given time period. Although the details will be described below, in the present embodiment, one index specified by the user, out of three indices, is to be able to be estimated from the other two indices based on an energy balance relation.

The model parameter determination unit 26 determines model parameters of estimation models used for estimating calories taken, calories consumed, and a change in the amount of calories stored. Here, the estimation models are terms of the following equation (1), respectively. The energy balance relation of ingestion, consumption, and storage using the estimation models is expressed as in the following equation (1).

κCᵢₙ(aᵢ, S)dt=∫Cₒᵤₜ(aₐ, xₒ)dt+∫C_{body}(a_{b}, x_{b})dt (1)

Here, Cᵢₙ denotes calories taken, Cₒᵤₜ denotes calories consumed, and C_{body} denotes a change in the amount of calories stored. Here, aᵢ denotes a model parameter of an estimation model for calories taken (hereinafter referred to as an intake calorie estimation model parameter), aₒ denotes a model parameter of an estimation model for calories consumed (hereinafter referred to as a consumed calorie estimation model parameter), and a_{b} denotes a model parameter of an estimation model for a change in the amount of calories stored (hereinafter referred to as a stored calorie change estimation model parameter). S denotes a meal feature vector determined from a change in a heart rate measurement, xₒ denotes an activity feature vector determined from an acceleration measurement, input height and weight values, and the like, and x_{b} denotes a weight and body-composition change feature vector determined from changes in a weight measurement, bio-impedance, input height value, and the like. In paragraphs of a specification, it is not possible to present alphabetical characters denoting vectors in bold type due to restrictions of the electronic filing system. Therefore, vectors are denoted by “S”, “a”, and “x”, which are alphabetical characters. In the present embodiment, the meal feature vector S is the area of a second heart-rate peak at the time of meal intake. The details of the area of the second heart-rate peak at the time of meal intake will be described below. The models, parameters, and features used for estimating calories consumed and for estimating a change in the amount of calories stored may be those used in estimation ways generally disclosed.

The model parameter determination unit 26 determines in advance two of the model parameters aᵢ, aₒ, and a_{b} of the above equation (1), and then determines (estimates) the remaining one model parameter (an unknown model parameter) based on S, xₒ, and xₒ obtained by measurement or estimation. The respective values of the model parameters aᵢ, aₒ, and a_{b} are stored in the model parameter storage unit 42.

The information presentation unit 28 determines information to be presented (recommended) to the user based on calories taken, calories consumed, and a change in the amount of calories stored that have been estimated by the intake calorie estimation unit 242, the consumed calorie estimation unit 244, and the stored calorie estimation unit 246. The information presentation unit 28 displays the determined information on the display unit 93 to present the information to the user.

The information storage unit 40 stores therein information acquired by the information acquisition unit 22 and other information.

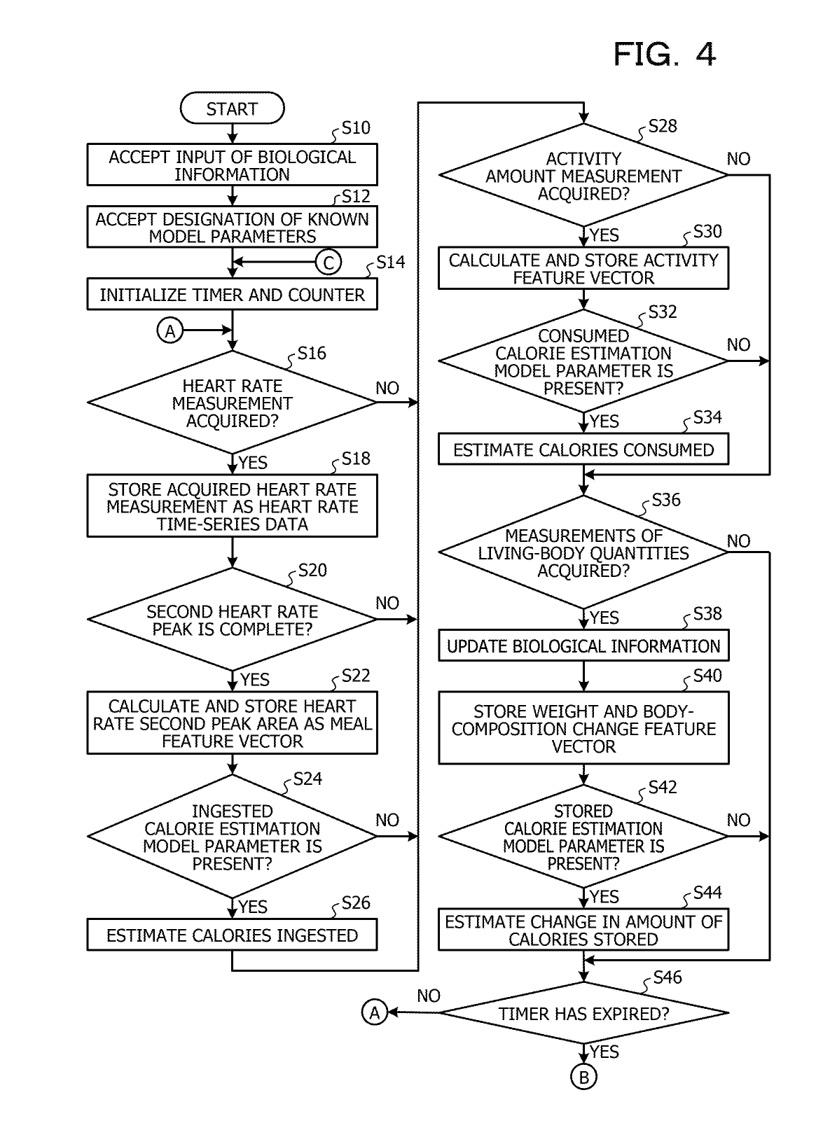

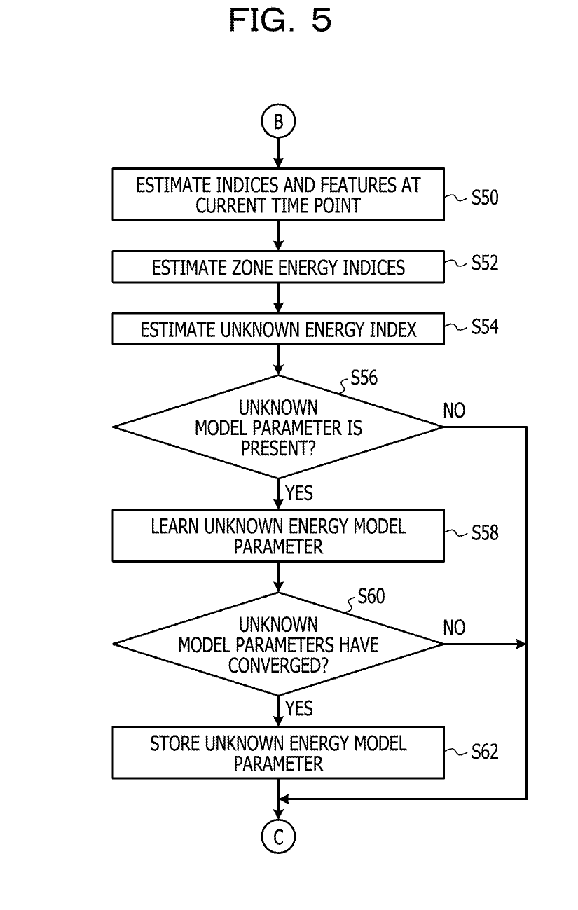

Next, the process of the information processing apparatus 20 of the present embodiment will be described in detail in accordance with flowcharts of FIG. 4 and FIG. 5.

In the process illustrated in FIG. 4, first, in S10, the information acquisition unit 22 accepts input of biological information. In this case, the information acquisition unit 22 accepts and acquires information entered by the user via the information processing apparatus 16 and the input unit 95. Here, the information entered by the user is, among biological information, information with very small changes over a long time period (age, height) and the initial values of biological information (initial value of weight, and the like). The information acquisition unit 22 stores the acquired information in the information storage unit 40. Instead of the age, the user may enter the birthday. In this case, the information acquisition unit 22 may calculate the age based on the birthday.

Next, in S12, the information acquisition unit 22 accepts designation of known model parameters. In this case, the information acquisition unit 22 accepts and acquires information about which two model parameters out of three model parameters aᵢ, aₒ, and a_{b}, are the model parameters designated as known model parameters by the user via the information input device 16 or the input unit 95. The model parameter determination unit 26 is to accept and acquire two known model parameters as user inputs or to use parameters determined in advance as default parameters of the two known model parameters, and is to learn the remaining one unknown model parameter as described below. Here, the known model parameters may be selected depending on, as determination criteria, for example, whether a model with good estimation accuracy is established, whether the difference between individuals is small, whether the measurement cost is low, and the like. Information about which of the model parameters are known model parameters and about which of the model parameters is an unknown model parameter and the acquired model parameters are transmitted to the energy index estimation unit 24 and the model parameter determination unit 26.

Next, in S14, the energy index estimation unit 24 initializes a timer and a counter. Specifically, the energy index estimation unit 24 resets, to zero, a timer t for measuring a time period (a given time period T) in which measurements used for estimation of calories taken, calories consumed, and a change in the amount of calories stored are acquired. Further, the energy index estimation unit 24 sets a counter n indicating the number of meals to zero.

Next, in S16, the information acquisition unit 22 determines whether a heart rate measurement has been acquired. If the determination in S16 is negative, that is, if no heart rate measurement has been acquired from the heart rate monitor 10, the process skips S18 to S26 and proceeds to S28. However, if this determination is affirmative, the process proceeds to S18.

When the process proceeds to S18, the information acquisition unit 22 stores the acquired heart rate measurement as heart rate time-series data in the information storage unit 40.

In S20, the information acquisition unit 22 determines whether the second heart rate peak is complete. If the determination in S20 is negative, the process skips S22 to S26 and proceeds to S28. However, if this determination is affirmative, the process proceeds to S22.

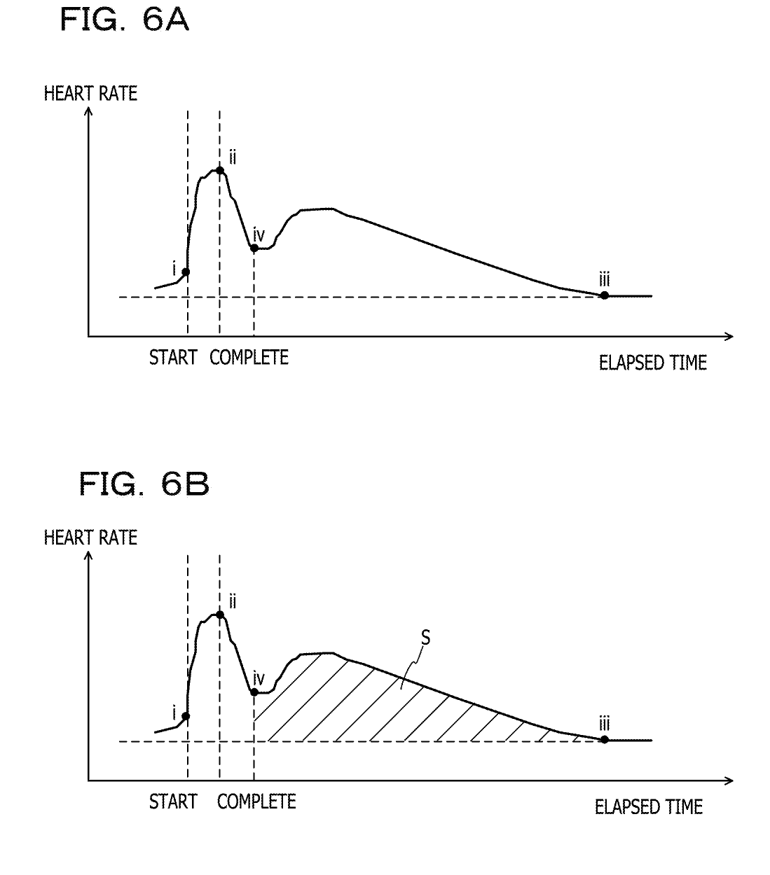

Here, the second heart rate peak will be described. FIG. 6A is a graph depicting an example of changes with time in heart rate (heart rate time-series data) derived from a meal. In FIG. 6A, the horizontal axis represents the elapsed time, and the vertical axis represents the heart rate. The heart rate refers to the number of heartbeats per unit of time, and is specifically the number of heartbeats per minute.

As illustrated in FIG. 6A, two peaks appear in a rising zone in which there is a rise in heart rate derived from a meal. The first peak is a change in heart rate that appears immediately after a meal starts, and the second peak is a change in heart rate that appears over a long period of time from the meal time. For example, the first peak is presumed to be derived from chewing, swallowing, motions of hands, or the like, and the second peak is presumed to be derived from motions of the stomach and intestines for digestion activities, absorption, and the like. The start time point of the rising zone at which the rise of the first peak is detected is a meal start time point i. A point ii of the maximum of the first peak is the meal completion time point. A time point iii at which the heart rate of the second peak returns to a given value after passing through the point of the maximum is the completion time point of the rising zone with the rise in heart rate derived from the meal. The time point at which a drop in heart rate settles down in the first peak is a time point iv. The given value may be the heart rate at the time at which a meal starts, or may be a value obtained by adding a certain value to the heart rate at the meal start time point or multiplying this heart rate by the certain value.

That is, in S20, the information acquisition unit 22 determines whether the time point iii appears in the heart rate time-series data.

If the determination in S20 is affirmative, the process proceeds to S22, where the intake calorie estimation unit 242 calculates and stores in the information storage unit 40 a heart rate second peak area S as a meal feature vector. Here, the heart rate second peak area S includes the integral of the biological response signal value changing with time after completion of a meal. Specifically, the heart rate second peak area S indicates the area S that is hatched in the illustration of FIG. 6B. That is, the area S of the second peak is the area between the time point iv at which a drop in heart rate has settled (the descending rate has decreased) in the first peak after completion of a meal and the time point iii. The intake calorie estimation unit 242 stores a calculation result of the heart rate second peak area S and a value (n=n+1) obtained by incrementing the counter n indicating the number of meals by one, together with the time, in the information storage unit 40.

Next, in S24, the intake calorie estimation unit 242 determines whether an intake calorie estimation model parameter is present, that is, whether the intake calorie estimation model is known. If the determination in S24 is negative, the process skips S26 and proceeds to S28. However, this determination is affirmative, the process proceeds to S26.

When the process proceeds to S26, the intake calorie estimation unit 242 estimates calories taken. Specifically, using the known intake calorie estimation model parameter aᵢ, the intake calorie estimation unit 242 estimates calories taken Cᵢₙ (aᵢ, S). The intake calorie estimation unit 242 stores the estimated calories taken, together with the time, in the information storage unit 40. Then, the process proceeds to S28.

When the process proceeds to S28, the information acquisition unit 22 determines whether an activity amount measurement has been acquired. If the determination in S28 is negative, that is, if no activity amount measurement has been acquired, the process skips S30 to S34 and proceeds to S36. However, if this determination is affirmative, the process proceeds to S30.

In S30, the consumed calorie estimation unit 244 calculates and stores in the information storage unit 40 an activity feature vector. In the present embodiment, the activity feature vector xₒ is assumed to be the amount of activity. The consumed calorie estimation unit 244 stores the activity feature vector xₒ and biological information, together with the time, in the information storage unit 40.

Next, in S32, the consumed calorie estimation unit 244 determines whether a consumed calorie estimation model parameter is present, that is, whether a consumed calorie estimation model parameter is known. If the determination in S32 is negative, the process skips S34 and proceeds to S36. Otherwise, if the determination in S32 is affirmative, the process proceeds to S34.

When the process proceeds to S34, the consumed calorie estimation unit 244 estimates calories consumed. Specifically, using a predetermined model, the consumed calorie estimation unit 244 estimates calories consumed Cₒᵤₜ (aₒ, xₒ) from the consumed calorie estimation model parameter aₒ and the activity feature vector xₒ. Further, the consumed calorie estimation unit 244 stores the estimated calories consumed, together with the time, in the information storage unit 40. Then, the process proceeds to S36.

When the process proceeds to S36, the information acquisition unit 22 determines whether the information acquisition unit 22 has acquired measurements of living-body quantities. If the determination in S36 is negative, that is, if no measurements of living-body quantities have been acquired, the process skips S38 to S44 and proceeds to S46. However, if this determination is affirmative, the process proceeds to S38.

When the process proceeds to S38, the information acquisition unit 22 updates biological information (for example, weight) stored in the information storage unit 40. If there is no change in biological information, the value of biological information in the information storage unit 40 is maintained as is.

Next, in S40, the stored calorie estimation unit 246 stores a weight and body-composition change feature vector in the information storage unit 40. In this case, the stored calorie estimation unit 246 stores the weight and body-composition change feature vector xₒ and biological information, together with the time, in the information storage unit 40.

Next, in S42, the stored calorie estimation unit 246 determines whether a stored calorie estimation model parameter is present, that is, whether the stored calorie estimation model parameter is known. If the determination in S42 is negative, the process skips S44 and proceeds to S46. However, if this determination is affirmative, the process proceeds to S44.

When the process proceeds to S44, the stored calorie estimation unit 246 estimates a change in the amount of calories stored. Specifically, using a predetermined model, the stored calorie estimation unit 246 estimates a change in the amount of calories stored C_{body} (a_{b}, x_{b}) from the stored calorie estimation model parameter a_{b} and the weight and body-composition change feature vector x_{b}. The stored calorie estimation unit 246 stores the estimated change in the amount of calories stored, together with the time, in the information storage unit 40. Then, the process proceeds to S46.

When the process proceeds to S46, the information acquisition unit 22 determines whether the timer t has expired (t=T), that is, whether the given time period T has elapsed. If the determination in S46 is negative, the process returns to S16 and repeatedly executes the processing and determinations of S16 to S44. Otherwise, if the determination in S46 is affirmative, the process proceeds to S50 in FIG. 5. At the stage where the process proceeds to S50, one of the three model parameters aᵢ, aₒ, and a_{b} is unknown. Therefore, two among the calories intake, the calories consumed, and the change in the amount of calories stored have been successfully estimated, and one is unknown. At the stage where the process proceeds to S50, the meal feature vector S per meal obtained in the given time period T, various measurements xₒ that are to be used for estimating consumed calories and that have been obtained in the given period T, and various measurements x_{b} that are to be used for estimating a change in the amount of calories stored obtained in the predetermined period T have been stored in the information storage unit 40.

When the process proceeds to S50, the model parameter determination unit 26 estimates an energy index (calories) and an energy index feature (S, xₒ, x_{b}) at the current time point. Specifically, based on the two types of energy indices and three types of energy index features obtained thus far, two types of indices and three types of energy index features at the current time point are estimated by extrapolation, and are stored, together with the time, in the information storage unit 40.

Next, in S52, the model parameter determination unit 26 estimates zone energy indices. In this case, the model parameter determination unit 26 integrates the energy indices of each time point to calculate energy indices in the given time period T, and stores the calculated energy indices in the information storage unit 40. Here, the above equation (1) may be specifically expressed as in the following equation (2). For the right-hand side of equation (2), details are omitted, and conceptual integral expressions are used. However, the expression of adding up values that have been stored for times, respectively, is given in reality.

$${\sum }_{k = 1}^{n} {C}_{in} ( {a}_{i} , {S}_{k} ) = {\int }_{0}^{T} {C}_{out} ( {a}_{o} , {x}_{o} ) dt + {\int }_{0}^{T} {C}_{body} ( {a}_{b} , {x}_{b} ) dt \qquad ( 2 )$$

In the above equation (2), the left-hand side represents the sum of calories taken in a given time period. On the left-hand side, n denotes the number of meals in the given time period, and k denotes the number of a meal among the meals taken in the given time period. The first term on the right-hand side represents the sum of calories consumed in the given time period. The second term on the right-hand side represents the sum of changes in the amount of calories stored in the given time period, that is, a change in the amount of calories stored from the start to the end of the given time period. That is, in S52, two among the left-hand side, the first term of the right-hand side, and the second term of the right-hand side in the above equation (2) are calculated.

Next, in S54, the model parameter determination unit 26 estimates an unknown energy index. In this case, an unknown energy index is estimated based on the two known zone energy indices and the above equation (2), (energy taken in=energy consumed+a change in the amount of energy stored). Thus, all of the energy taken, energy consumed, and a change in the amount of energy stored in the given time period have been successfully estimated. The model parameter determination unit 26 stores the estimated energy indices in the information storage unit 40.

Next, in S56, the model parameter determination unit 26 determines whether an unknown model parameter is present. If the determination in S56 is negative, the process returns to S14 and repeats the processing and determinations of S14 to S54. Otherwise, if the determination in S56 is affirmative, the process proceeds to S58.

When the process proceeds to S58, the model parameter determination unit 26 performs learning of an unknown energy model parameter. In this case, the model parameter determination unit 26 uses an unknown energy index stored in the information storage unit 40 (energy index estimated in S54) and an energy index feature corresponding to the unknown energy index to determine an unknown model parameter, and temporarily stores the unknown model parameter.

Next, in S60, the model parameter determination unit 26 determines whether unknown model parameters have converged. In this case, if a difference between the value of the current unknown model parameter and each of the values of a given number of unknown model parameters determined most recently is within a predetermined range, the determination in S60 is affirmative, and the process proceeds to S62. Otherwise, if the determination in S60 is negative, the process returns to S14 and repeats the processing and determinations of S14 to S58.

If the determination in S60 is affirmative, that is, if the values of unknown model parameters have converged, the process proceeds to S62. Further, the model parameter determination unit 26 stores the value of the unknown model parameter (the value temporarily stored in S58) in the model parameter storage unit 42.

Thereafter, the process returns to S14 and repeats the processing of S14 and the subsequent steps. After storing the unknown model parameter in the model parameter storage unit 42, the information processing apparatus 20 may also perform the processing of learning an unknown model parameter at a suitable timing. The information processing apparatus 20 may set the model parameter determined as being known in the above processing to be unknown and also perform the processing of learning the unknown model parameter.

In a situation where, as described above, the process illustrated in FIG. 4 and FIG. 5 is executed, so that all of the three model parameters aᵢ, aₒ, and a_{b} are present, calculation or estimation of any of S, xₒ, and x_{b} may be omitted. That is, when acquisition of, for example, x_{b} is omitted, calories taken are estimated using aᵢ and S and calories consumed are estimated using aₒ and xₒ, and then a change in the amount of calories stored may be estimated based on the above equation (2) (calorie balance relation). When acquisition of, for example, xₒ is omitted, calories taken are estimated using aᵢ and S and a change in the amount of calories stored is estimated using a_{b} and x_{b}, and then calories consumed may be estimated based on the above equation (2). Further, when acquisition of, for example, S is omitted, calories consumed are estimated using aₒ and xₒ and a change in the amount of calories stored is estimated using a_{b} and x_{b}, and then calories taken may be estimated based on the above equation (2). In such a manner, omitting calculation or estimation of any of S, xₒ, and x_{b} enables energy indices of the user to be easily estimated. Here, when two energy index features to be actually calculated or estimated are selected from among the energy index features S, xₒ, and x_{b}, it may be used, as a determination criterion, for example, whether a model with good estimation accuracy is established or whether the measurement cost is low. For example, since S is able to be determined based on heart rates, and the measurement cost for heart rates is lower than for another item, it is possible to select S as an object to be measured.

When a complex estimation model is determined as an estimation model, the number of elements of an unknown model parameter is sometimes large. In such a case, after elapse of a plurality of time periods, error function=∥calories taken−(change in the amount of calories stored+calories consumed)∥ is generated for each of the plurality of time periods, and the value of the unknown model parameter that minimizes the total sum of error functions of all the periods may be searched for and determined.

Next, the presentation processing of the information presentation unit 28 will be described. Here, a meal is one of elements that greatly affect human health, and therefore a proper meal has to be taken. For example, when a meal that adversely affects the health is continuously taken, attention has to be called for. When the value of calories consumed is low, exercise has to be encouraged for health. Accordingly, in the present embodiment, based on indices related to energy use of a living body estimated in everyday life (calories taken Cᵢₙ, calories consumed Cₒᵤₜ, and a change in the amount of calories stored C_{body}), the information presentation unit 28 determines information as described below as information to be presented to the user. Information other than Cᵢₙ, Cₒᵤₜ, and C_{body}, for example, time information, information about a habit of the user registered in advance by himself or herself, and the like may be accounted for in determining the information to be presented.

(1) In the case where Cᵢₙ-Cₒᵤₜ>threshold, the information presentation unit 28 determines information indicating overeating as information to be presented.

(2) The information presentation unit 28 converts ∥Cᵢₙ−Cₒᵤₜ∥ to the number of steps, and determines information that encourages the user to walk by the number of steps obtained by the conversion, as information to be presented.

(3) In the case where Cᵢₙ−Cₒᵤₜ<threshold, meal intake interval>threshold, and the time is within a specific range, the information presentation unit 28 determines information that encourages the user to take a meal as information to be presented.

(4) In the case where Cᵢₙ−Cₒᵤₜ>threshold, C_{body}>threshold, and Cₒᵤₜ>threshold, the information presentation unit 28 determines information indicating that the current amount of exercise is appropriate, as information to be presented.

(5) In the case where Cᵢₙ−Cₒᵤₜ>threshold and Cᵢₙ>threshold, the information presentation unit 28 determines information indicating that the user eats too much and has to increase the amount of exercise, as information to be presented.

(6) In the case where Cᵢₙ−Cₒᵤₜ>threshold and Cₒᵤₜ>threshold, the information presentation unit 28 determines information indicating that the amount of exercise is excessive and the user has to reduce the amount of meal instead of reducing the amount of exercise, as information to be presented.

As described above, the information presentation unit 28 determines information to be presented based on the calories taken Cᵢₙ, the calories consumed Cₒᵤₜ, the change in the amount of calories stored C_{body} in a given time period, thereby enabling appropriate information about meals and exercise to be presented to the user.

The foregoing method to determine the information to be presented is an example. That is, information to be presented may be determined based on a ratio between two energy indices (calories).

In the foregoing, the case where energy indices are expressed in units of calories (heat values) has been described. However, other values (for example, weights (g) of carbohydrates) may be used if the values are energy-related values and are able to be substituted in the energy balance equation (equation (1)) when their units are converted.

In the foregoing, the case where the energy indices are three indices, calories taken, calories consumed, and a change in the amount of calories stored, has been described; however, the energy indices are not limited to this. For example, one of all the indices may be set as an unknown energy index on the assumption that each of three energy indices includes a plurality of indices as follows:

(a) calories taken=calories of carbohydrates+calories of fat+calories of protein,

(b) calories consumed=activity induced energy expenditure+basal metabolism+diet induced thermogenesis+non-exercise activity thermogenesis, and

(c) a change in the amount of calories stored=a change in the amount of calories stored resulting from fat+a change in the amount of calories stored resulting from the liver+a change in the amount of calories stored resulting from muscle.

Next, a method for estimating an intake calorie estimation model parameter will be described in more detail.

It is supposed that calories taken Cᵢₙ at the time of meal intake and the heart rate second peak area S at that time are able to be modeled as in the following equation (3).

Cᵢₙ−Cₕₑₐₜ=f(a, S) (3)

Here, Cₕₑₐₜ denotes diet induced thermogenesis, and a denotes an intake calorie estimation model parameter. Diet induced thermogenesis, which is included in calories consumed Cₒᵤₜ, may be included in a model of calories taken because the calories taken are used as a variable in a typical estimation model.

The calorie balance relation may be given by the following equation (4).

Cᵢₙ−Cₕₑₐₜ=C_{Δbd}+C_{act}+C_{bm} (4)

Here, C_{Δbd} denotes calories stored as fat or muscle, C_{act} denotes calories consumed by an activity, and C_{bm} denotes calories consumed by basal metabolism.

Assuming that n meals are taken in the given time period T, the total calories taken in the given time period T may be given by the following equation (5).

$${\int }_{0}^{T} ( {C}_{in} - {C}_{heat} ) dt = {\sum }_{k = 1}^{n} f ( a , {S}_{k} ) \qquad ( 5 )$$

The total calories consumed in the given time period T may be given by the following equation (6).

∫₀^{T}(C_{Δbd}+C_{act}+C_{bm})dt=∫₀^{T}C_{Δbd}dt+∫₀^{T}C_{act}dt+∫₀^{T}C_{bm}dt (6)

Here, it is possible to calculate the first term on the right-hand side of the above equation (6), for example, by multiplying a weight difference between the start and the end of the given time period T by a constant. It is possible to calculate the second term on the right side, for example, by integrating the calories consumed that have been measured by the activity tracker 12 in the given time period T. It is possible to calculate the third term on the right side, for example, from the averages of weights, heights, and ages between the start and the end of the given time period T and gender information by using Harris-Benedict Equation or the like.

Given that the value on the right-hand side (the sum of the first, second, and third terms) of the above equation (6) obtained in this way is C_{T}, the relationship of the next equation (7) is obtained from the above equations (5) and (6).

$${\sum }_{k = 1}^{n} f ( a , {S}_{k} ) = {C}_{T} \qquad ( 7 )$$

Further, the difference between both sides of the above equation (7) in a certain time period m is expressed as in the next equation (8), and Sₘ, C_{Tm}, and nₘ obtained by measurement or the like are substituted in this equation, so that a that minimizes ∥gₘ(a, Sₘ, C_{Tm})∥ is estimated as the intake calorie estimation model parameter.

$${g}_{m} ( a , {S}_{m} , {C}_{Tm} ) = {\sum }_{k = 1}^{{n}_{m}} f ( a , {S}_{mk} ) - {C}_{Tm} \qquad ( 8 )$$

By a calculation as described above, it is possible to estimate an intake calorie estimation model parameter.

Here, in the case where there are a plurality of elements of a, the weight gain or loss or the like on the right-hand side of the above equation (6) is discretely measured, so that the result is to be obtained as a plurality of relationships in each time period m of the above equation (8). For example, a that minimizes the following equation (9) may be obtained.

$${\sum }_{m = 1}^{M} \vert {g}_{m} ( a , {S}_{m} , {C}_{Tm} , {n}_{m} ) \vert = {\sum }_{k = 1}^{M} \vert {\sum }_{k = 1}^{{n}_{m}} f ( a , {S}_{mk} ) - {C}_{Tm} \vert \qquad ( 9 )$$

Cₕₑₐₜ is typically handled as a quantity proportional to Cᵢₙ, and its proportional coefficient is said to differ according to the kind of nutrient taken. Supposing that a rise in heart rate due to meal intake exhibits variations that differ according to taken nutrients, when a plurality of nutrients are taken, the rise may be supposed as a result of synthesis of their respective variation components.

Accordingly, the heart rate second peak area S and the proportional coefficient α are set as L areas and proportional coefficients for each nutrient, and the model of the above equation (3) may be defined as given by the following equation (10).

$${C}_{in} - \frac{{\sum }_{l = 1}^{L} {α}_{l} {S}_{l}}{{\sum }_{l = 1}^{L} {S}_{l}} {C}_{in} = f ( a , {S}_{1} , {S}_{2} , … , {S}_{l} ) \qquad ( 10 )$$

Modifying the above equation (10) gives the following equation (11).

$$\begin{matrix}{C}_{in} = \frac{{\sum }_{l = 1}^{L} {S}_{l}}{{\sum }_{l = 1}^{L} ( 1 - α ) {S}_{l}} f ( a , {S}_{1} , {S}_{2} , … , {S}_{l} ) \\ = {f}_{h} ( a , {S}_{1} , {S}_{2} , … , {S}_{l} )\end{matrix} \qquad ( 11 )$$

Here, a is a constant determined in advance. Accordingly, as in the above equation (11), f of the above equation (3) is defined as fₕ including the effects of diet induced thermogenesis, thereby enabling the intake calorie estimation model parameter a to be determined as described above.

Next, a method for estimating an intake calorie estimation model parameter in the case where the relationship between the calories taken at the time of meal intake and the heart rate second peak area S at that time is a linear relationship will be described.

In this case, diet induced thermogenesis has a proportional relationship to calories taken, and therefore the above equation (3) may be expressed as in the following equation (12).

Cᵢₙ−Cₕₑₐₜ=Cᵢₙ−αCᵢₙ=vS+w (12)

Here, v denotes a slope coefficient, w denotes an intercept coefficient, α denotes a proportional coefficient (for example, 0.1).

The calorie balance relation in this case is expressed by the above equation (4). Therefore, assuming that n meals are taken in the given time period T, the total calories taken in the given time period T may be expressed by the following equation (13).

$${\int }_{0}^{T} ( {C}_{in} - {C}_{heat} ) dt = {\sum }_{k = 1}^{n} ( {vS}_{k} + w ) = v {\sum }_{k = 1}^{n} {S}_{k} + nw \qquad ( 13 )$$

ΣSₖ sums S detected at the time of each meal intake in the given time period T, enabling the result to be obtained as S_{T}.

The total calories consumed in the given time period T may be expressed by the above equation (6). Given that the values on the right-hand side (total sum of the first, second, and third terms) of the above equation (6) is C_{T}, the relationship of the following equation (14) is obtained from the above equations (13) and (6).

vS_{T}+nw=C_{T} (14)

Further, the given time period T is determined, and processing of measuring or calculating the number of meals n in the period T, S_{T}, and C_{T} is performed for a plurality of time periods. As a result, a plurality of sets of n, S_{T}, and C_{T}, are acquired. Further, the coefficients v and w of vS_{T}+nw=C_{T} are obtained, for example, by linear regression.

Calculation as described above enables an intake calorie estimation model parameter to be estimated in the case where the relationship between the calories taken at the time of meal intake and the heart rate second peak area S at that time is a linear relationship.

In the above example, the case where the given time period T is determined, and processing of measuring or calculating the number of meals n in the time period T, S_{T}, and C_{T} is performed for a plurality of time periods has been described; however, the present embodiment is not limited to this. For example, the number of meals N is determined in advance, and processing of measuring or calculating S_{T} and C_{T} may be performed for a plurality of time periods in each of which a meal intake occurs N times. In this case, from a plurality of sets of S_{T} and C_{T} obtained, the coefficient V and W of VS_{T}+W=C_{T} are determined, for example, by linear regression. At this point, since V=v and W=Nw from the above equation (14), it is possible to determine the coefficient of the above equation (3).

As described in detail in the above, according to the present embodiment, by using estimation models, the energy index estimation unit 24 estimates indices other than one unknown index, out of three indices: an index regarding energy taken by a living body in the given time period T (calories taken Cᵢₙ), an index regarding energy consumed by the living body in the given time period T (calories consumed Cₒᵤₜ), and an index regarding a change in the amount of energy stored in the living body in the given time period T (a change in the amount of calories stored C_{body}); and the model parameter determination unit 26 estimates the unknown index based on a relation satisfied by the three indices (calories taken=calories consumed+a change in the amount of calories stored) and the estimation result of indices other than the unknown index (S54). Further, the model parameter determination unit 26 acquires, in advance, two of the model parameters aᵢ, aₒ, and a_{b} of estimation models and generates the remaining one based on the model parameters acquired in advance and the estimated values of three indices, Cᵢₙ, Cₒᵤₜ, and C_{body} (S58). An index regarding energy taken by a living body is an index related to a feature of a change with time of a biological response signal value derived from a meal after completion of the meal (the heart rate second peak area S in the present embodiment). Thus, in the present embodiment, since an unknown model parameter is determined based on the three estimated indices, Cᵢₙ, Cₒᵤₜ, and C_{body}, model parameters may be more easily determined than in the case where three model parameters are determined based on measurements or the like. In the present embodiment, since, based on any two among the calories taken Cᵢₙ estimated based on the heart rate second peak area S, the calories consumed Cₒᵤₜ, and the change in the amount of calories stored C_{body}, the remaining one is estimated, an energy index feature (personal biological information or the like) used for estimating the remaining one does not have to be acquired. Accordingly, it is possible to easily estimate indices regarding three types of energy.

In the present embodiment, the information presentation unit 28 presents information regarding a living body to the user based on at least one of the three indices, Cᵢₙ, Cₒᵤₜ, and C_{body}. This makes it possible to provide the user with information related to precaution regarding ingestion and consumption of calories and recommended activities. The information presentation unit 28 determines information to be presented based on differences among indices or the like and therefore is able to easily determine information to be presented.

In the present embodiment, since the heart rate second peak area S is used in estimating calories taken, it is possible to estimate the calories taken with high accuracy. In this case, without having to perform an activity for intake of a meal of known calories, the calories taken are able to be accurately estimated from the heart rate from a meal.

In the embodiment described above, description has been given of the case where the heart rate second peak area S is used in estimating calories taken; however, the present embodiment is not limited to this. For example, at least one of the rise rate and the drop rate between one point of a biological response signal value changing with time and one point of a biological response signal value changing with time after completion of a meal may be used. At least one of the rise width and the drop width between one point of a biological response signal value changing with time and one point of a biological response signal value changing with time after completion of a meal may also be used. The time width between one point of a biological response signal value changing with time and one point of a biological response signal value changing with time after completion of a meal may also be used.

The processing functions described above may be achieved by a computer. In that case, a program in which processing details of functions to be possessed by a processing apparatus are described is provided. When the program is executed by a computer, the processing functions described above are achieved on the computer. The program in which processing details are described may be recorded on a computer-readable recording medium (however, except for carrier waves).

When a program is distributed, for example, the program is sold in the form of a portable recording medium, such as a digital versatile disc (DVD) or a compact disc read-only memory (CD-ROM), on which the program is recorded. A program may also be stored in a storage device of a server computer and be transferred from the server computer to another computer.

A computer that executes a program stores, in a storage device thereof, for example, a program recorded on a portable recording medium or a program transferred from a server computer. Further, the computer reads the program from the storage device thereof and executes processing according to the program. The computer may also read a program directly from a portable recording medium and execute processing according to the program. Each time a program is transferred from a server computer, the computer may also sequentially execute processing according to the received program.

All examples and conditional language recited herein are intended for pedagogical purposes to aid the reader in understanding the invention and the concepts contributed by the inventor to furthering the art, and are to be construed as being without limitation to such specifically recited examples and conditions, nor does the organization of such examples in the specification relate to a showing of the superiority and inferiority of the invention. Although the embodiment of the present invention has been described in detail, it should be understood that the various changes, substitutions, and alterations could be made hereto without departing from the spirit and scope of the invention.

What is claimed is:

1. An information processing method executed by a processor included in an information processing apparatus coupled to a sensor, the information processing method comprising:
acquiring information on a living body from the sensor;
estimating, by using estimation models, indices other than one unknown index among three indices including an index being related to a feature of a change with time of a biological response signal value derived from a meal after completion of the meal, an index regarding energy consumed by the living body in a predetermined time period, and an index regarding a change in an amount of energy stored in the living body in the predetermined time period, based on information received from the sensor;
estimating the one unknown index based on a relation satisfied by the three indices and a result of the estimating indices; and
generating at least one of model parameters of the estimation models based on estimated values of the three indices.

2. The information processing method according to claim 1, wherein the generating includes:
acquiring a model parameter other than at least one unknown model parameter, out of the model parameters of the estimation models, and
determining the at least one unknown model parameter based on the acquired model parameter and the estimated values of the three indices.

3. The information processing method according to claim 1,
wherein the estimating indices other than one unknown index includes estimating indices other than the unknown index by using the estimation models based on an actual measured value or an input value for the living body.

4. The information processing method according to claim 1, further comprising
outputting information on the living body based on at least one of the three indices.

5. The information processing method according to claim 4, wherein
the index being related to the feature of the change with time of the biological response signal value derived from the meal after completion of the meal is used to calculate an index regarding energy taken by the living body, and
the information on the living body is information based on at least a difference or a ratio between the index regarding energy taken by the living body and the index regarding energy consumed by the living body.

6. The information processing method according to claim 1,
wherein the feature includes an integral of the biological response signal value changing with time after completion of a meal.

7. The information processing method according to claim 1,
wherein the three indices characterize an energy balance relation of the living body.

8. The information processing method according to claim 1, wherein the three indices characterize an energy balance relation of the living body and the method further comprises:
outputting information to the living body identifying a status of living body based on the energy balance relation, the status being the living body has overeaten, exercise is needed, a meal is needed, balanced, exercise is excessive.

9. The information processing method according to claim 1, wherein the three indices are components of an energy balance relation of the living body represented by the following equation
∫Cᵢₙ(aᵢ, S)dt=∫Cₒᵤₜ(aₐ, xₒ)dt+∫C_{body}(a_{b}, x_{b})dt (1)
wherein Cᵢₙ denotes calories taken, Cₒᵤₜ denotes calories consumed, and C_{body} denotes a change in the amount of calories stored in the living body,
aᵢ denotes a model parameter of an estimation model for calories taken, aₒ denotes a model parameter of an estimation model for calories consumed, and a_{b} denotes a model parameter of an estimation model for a change in the amount of calories stored in the living body,
S denotes a meal feature vector determined from a change in a heart rate measurement measured by the sensor, xₒ denotes an activity feature vector, and x_{b} denotes a weight and body-composition change feature vector.

10. The information processing method according to claim 6, wherein
the estimating the index being related to a feature of a change with time of a biological response signal value derived from a meal after completion of the meal includes:
monitoring a heart rate of the living body during period associated with the meal, the period associated with the meal including first period including a rising heart rate immediately after the meal starts and a second period with a rising heart rate after the first period,
identifying a point between the first period and the second period as the completion of the meal, and
calculating the integral of the biological response signal value during the second period of the period associated with the meal.

11. An information processing system comprising:
a sensor configured to acquire information on a living body; and
an information processing apparatus coupled to the sensor and including a processor
wherein the processor is configured to:
estimate, by using estimation models, indices other than one unknown index among three indices including an index being related to a feature of a change with time of a biological response signal value derived from a meal after completion of the meal, an index regarding energy consumed by the living body in a predetermined time period, and an index regarding a change in an amount of energy stored in the living body in the predetermined time period, based on information received from the sensor;
estimate the unknown index based on a relation satisfied by the three indices and a result of the estimating; and
generate at least one of model parameters of the estimation models based on estimated values of the three indices.

12. A computer-readable recording medium storing a program that causes a processor included in an information processing apparatus coupled to a sensor to execute a process, the process comprising:
acquiring information on a living body from the sensor;
estimating, by using estimation models, indices other than one unknown index among three indices including an index being related to a feature of a change with time of a biological response signal value derived from a meal after completion of the meal, an index regarding energy consumed by the living body in a predetermined time period, and an index regarding a change in an amount of energy stored in the living body in the predetermined time period, based on information received from the sensor;
estimating the one unknown index based on a relation satisfied by the three indices and a result of the estimating indices; and
generating at least one of model parameters of the estimation models based on estimated values of the three indices.
